(19) United States
(12) Patent Application Publication
(10) Pub. No.: US 2008/0188740 A1
(43) Pub. Date: Aug. 7, 2008

(54) APPARATUS AND METHOD FOR GUIDING CATHETERS

(76) Inventors: Cesar M. Diaz, Rancho Santa Margarita, CA (US); Kim McGurrin, Guelph (CA); Peter Physick-Sheard, Rockwood (CA)

Correspondence Address: WHITAKER, CHALK, SWINDLE & SAWYER, LLP, 3500 CITY CENTER TOWER II, 301 COMMERCE STREET, FORT WORTH, TX, 76102-4186, US

(21) Appl. No.: 11/943,928

(22) Filed: Nov. 21, 2007

Related U.S. Application Data
(63) Continuation-in-part of application No. 11/602,860, filed on Nov. 21, 2006.
Application No. 11/602,860 is a continuation-in-part of application No. 11/363,361, filed on Feb. 27, 2006.
Application No. 11/363,361 is a continuation-in-part of application No. 10/757,948, filed on Jan. 14, 2004, now abandoned.

(51) Int. Cl. A61B 8/00

(52) U.S. Cl. 600/424

(57) ABSTRACT

The present invention is a system for guiding catheters into chamber or conduits of the body without the use of X-ray based imaging systems. The system disclosed is used for guidance of catheters in the heart chamber and heart protruding structures and conduits by using external ultrasound and device based physiological sensory inputs to create a quasi-visual-sensory-algorithm that is used to provide clinical sensory and handling input so that device placement is facilitated. The method and preferred devices are designed to deliver high energy defibrillation shocks to the myocardium and also provide a stable substrate for pressure lumens and or sensors used to provide “distal specific” physiological sensory inputs.

### CROSS REFERENCE TO RELATED APPLICATIONS

The present application is a continuation-in-part of earlier filed application Ser. No. 11/602,860, filed Nov. 21, 2006, entitled “Apparatus and Method for Guiding Catheters”, which was, in turn, a continuation-in-part of Ser. No. 11/363,361, filed Feb. 27, 2006, which was, in turn, a continuation-in-part of Ser. No. 10/757,948, filed Jan. 14, 2004, now abandoned.

### FIELD OF THE INVENTION

The present invention generally relates to devices and methods used to guide catheters into body lumens and chambers using a set of non-X ray based methods that include ultrasound and pressure gradients to form a quasi-visual-sensory-algorithm that can be used to guide the devices into a desired location within the body.

### DESCRIPTION OF THE PRIOR ART

Atrial Fibrillation is a cardiac disease that has been widely reported in humans throughout the world with patient population estimates ranging in the 6 to 10 million with an annual compound growth rate estimated at 7% per annum. The disease is complex and commonly associated with chaotic electrical disturbances found in the atria but originating from a variety of regions in and around the heart. The disease is further complicated by differences in underlying disease states such as structural heart disease and coronary artery disease. Considerable work has been done and apparatus and methods defined to terminate this disease using internal cardioversion by contributors including Levy, Dolla, Jaros, Alt, Accorti, Diaz, and others.

Most of the prior work concerns devices with utility in the electrophysiology laboratory. The devices for the most part define a sensing catheter that also delivers electrical energy to terminate “shockable” arrhythmias with less energy than an external defibrillator. However, early work done as part of the Rhythm Clinical Study for Cardiac Arrhythmias that was initiated in 1995 showed anecdotal evidence of a curative effect for some atrial fibrillation patients. However, at the time, the data were considered to be too inconsistent and unclear and possibly an artifact. The prevailing concern at the time was energy reduction driven by the desire to create a painless implantable internal cardiac defibrillator (ICD). The ICD science dominated the field's interest and also the minds of many of the researchers perfecting this technology. Work done by Mirowski (U.S. Pat. No. 3,616,955), Heilman (U.S. Pat. No. 4,270,549) and others achieved success at terminating singular events of cardiac arrhythmias on an as presented basis. The work by Diaz in provisional application, Ser. No. 60/451,005, filed Mar. 3, 2003 and utility application Ser. No. 10/757,948, filed Jan. 14, 2004, is aimed at describing configurations, methods and apparatus, including catheter/lead designs, that are ideally suited for coupling electrical energy to the heart muscle with sufficient efficiency that not only low and medium energy (0.5 to 30 Joules) but also high energy (over 30 Joules) can be coupled safely.

To test these concepts on an animal model, a good candidate had to be found with biological (natural) atrial fibrillation. One professional from the field was seeking a solution to the treatment of equine atrial fibrillation as an alternate or adjunctive treatment to drugs. These efforts lead to contact with one of the leading companies of such devices, Rhythm Technologies, Inc. The collaboration then expanded to include a company doing much of the development work for the Rhythm Technology devices, Polymer Component Services, Inc. or PolyComp (now Cardiac Output Technologies, Inc.). Over the last few years, a great deal of effort has been expended in an attempt to reduce theory to clinical practice. As a result, the parties have had to solve problems that are unique to catheter and lead technologies as well as equine medicine. For example, it was necessary to properly suit the catheter and electrodes to treat an equine animal. However, certain aspects of the procedure were so different that they have led to new and stand alone discoveries and inventions, particularly in the area of catheter guidance and also physiological monitoring during the procedure for safety and patient care.

The use of ultrasound devices to view inside an object and/or mammal is not a new concept. In U.S. Pat. No. 6,520,916, Brennen teaches that an ultra sound image can be captured from an indwelling device if the device is equipped with a vibrating stylet or mandrel. A Doppler image of the device can be recorded and used to locate the device housing the vibrating stylet. This and other work claims to facilitate the imaging of devices within an animal, as taught by King (U.S. Pat. No. 4,100,916), that work being originally pioneered by Rocha (U.S. Pat. No. 3,780,572), Glover (U.S. Pat. No. 4,075,883) and Heyser (U.S. Pat. No. 4,078,232). Additional relevant work by Daniels (U.S. Pat. No. 4,290,432) teaches that bubbles are, sufficiently different in density from tissue that detection can be observed and measurements made. Johnson (U.S. Pat. No. 3,710,615) also teaches the ability to measure varying densities within liquids and solids and discern, for example, the concentration of oil in water.

It therefore appears to be understood in the prior art that ultrasound can be used in soft tissue and that when a dense material is used within that tissue an image can be adequately collected and presented; such as of a surgical instrument. However, when one is trying to use flexible medical devices, primarily made of elastomers or plastics, within the body, a set of problems are encountered. The relative density differences are smaller and therefore the image is poor. Varying types of tissue such as bone, cartilage and fluid/air filled organs such as the lungs can further complicate imaging deep within the body. In order for a device to be used successfully, one must employ more than just the ultrasound image to guide the device into place. In fact the indwelling device itself must provide some feedback so that the ultrasound image can be used more effectively in instrument guidance. The present invention builds upon the teachings of Rocha, King, and Johnson to preferentially design devices with features that are conducive to ultrasound image enhancement and that are useful, in part, in navigating devices to specific locations.

### SUMMARY OF THE INVENTION

The present invention teaches the proper anatomical positioning of catheters used to treat equine atrial fibrillation. In the preferred form of the invention, a device and method are provided for “steering” and “guiding” one or more apparatuses deep into the body and more specifically into the heart. The method described is a multiple indicator/feedback approach to the placement of the devices and confirmation of proper placement using ultrasound, pressure gradients and depth measurements to guide devices to specific target locations.

The method is ideally suited to place the device into regions in and around the heart of a large mammal such as an equine animal and, more specifically, into the heart and anatomically linked body lumens such as the pulmonary artery and pulmonary veins for a variety of clinically relevant reasons. One preferred embodiment is the termination of atrial fibrillation using high energy defibrillation.

The present invention provides a series of steps and elements of a device which will allow catheters to be navigated into body lumens and cavities and more specifically, the heart. Measurements are taken from a single multi-function catheter or a set of indwelling catheters and combined with external measurements and are used to monitor the anatomical location of a device so as to provide a simple non x-ray based guidance system that can be manual or automatic, and is referred to as a quasi-visual-sensory-algorithm (Q-VSA).

In the method of equine cardioversion of the present invention a catheter is inserted into the jugular vein of the horse and then mechanically guided into the heart by way of mechanical displacement monitoring. At the same time, device-based pressure gradient measurements are used to confirm placement. Additional ultrasound imaging can be done to ensure proper location and placement of the catheter. In the next step of the method, device-based electrical cardioversion is then performed.

Additional objects, features and advantages will be apparent in the written description which follows.

### BRIEF DESCRIPTION OF THE DRAWINGS

FIG. 1 shows a preferred embodiment of catheter placement for the treatment of equine atrial fibrillation using a two catheter system with each catheter having a single high surface electrode, the catheter placement being shown with the heart base viewed from above.

FIG. 2 shows a preferred embodiment of a catheter fashioned with a pressure gradient lumen distal to a high surface electrode and just proximal of the distal tip.

FIG. 3 shows a preferred embodiment of a catheter fashioned with a dual pressure gradient lumen set-up so that one lumen is located distal to a high surface electrode, the other lumen being located proximal to the high surface electrode.

FIGS. 4A and 4B show a preferred embodiment of a catheter of the invention fashioned with an ultrasound enhancing ring located proximal to the high surface electrode for purposes of enhancing the ultrasound image.

FIG. 5 shows the distal tip of the catheter made of high density metal and preferably alloy of Noble metal and further equipped with an ultrasound reflective adhesive ring or bond joint affixing the distal tip to the catheter body; the design serving to enhance the ultrasound image and facilitate an additional or alternate intra-cavitary image system, such as an echosonograph.

FIGS. 6A and 6B shows the distal end of the catheter fashioned with an orientation marker designed to provide feedback on distal tip curve orientation and/or amount of deflection, if using active deflection, or catheter distal end when using ultrasound of echosonograph as guidance system.

FIGS. 7A-7C are graphical representations of pressure gradients taken from an indwelling device that is used to guide the catheter into the right atrium by use of mechanical displacement measurements and device-based indwelling pressure gradient monitoring.

FIGS. 8A-8C are similar graphical representations of pressure gradients taken from an indwelling device that is used to guide the catheter into the right atrium by use of mechanical displacement measurements and device-based indwelling pressure gradient monitoring.

FIG. 8D is a follow-on of the procedure graphically illustrated in FIG. 8A-8C with ultrasound then being used to ensure placement of the device is into left pulmonary artery.

FIGS. 9 and 10 illustrate an alternate catheter configuration that uses a single catheter, rather than dual catheters.

### DETAILED DESCRIPTION OF THE INVENTION

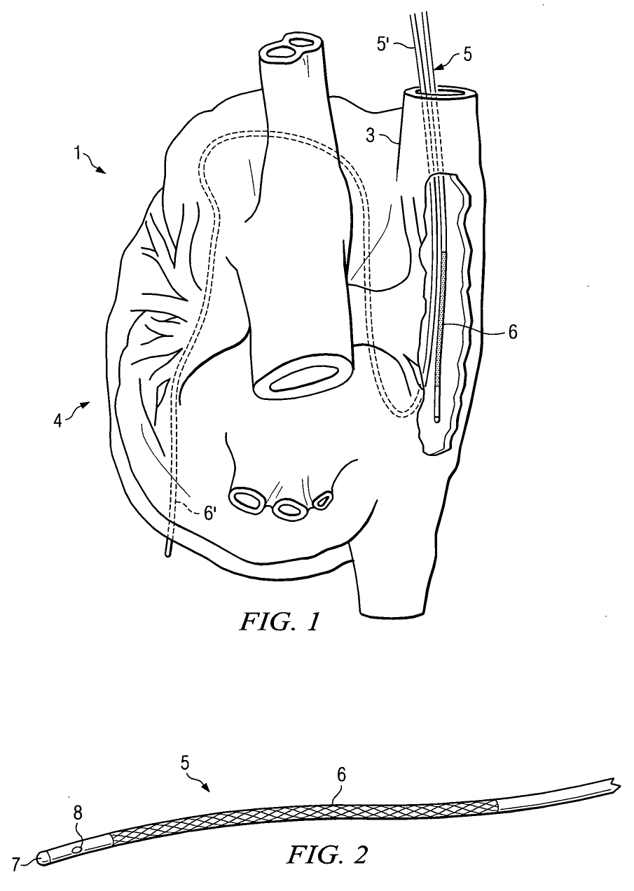

The present invention teaches the proper anatomical positioning of catheters, which, in the preferred form, are used to treat equine atrial fibrillation. FIG. 1 shows an image of the proper placement of catheters within the equine heart 1. A catheter (5) is placed in the right atrium (3), said catheter equipped with a high surface electrode (6) (see FIG. 2) that acts as a cathode/anode. A second catheter (5′) is advanced into the pulmonary artery (4) and more specifically the left pulmonary branch using the same mechanical and device-based pressure gradient guidance techniques, the catheter being equipped with a high surface electrode (6′), which acts as a cathode/anode.

The devices are designed to facilitate the technique and include several specific design attributes. FIG. 2 shows one preferred embodiment of the present invention in which a catheter (5) is equipped with a pressure sensing means (8) and a distal tip (7) that is visible by using ultrasound or X-ray, and also a high surface electrode (6). The high surface electrode is capable of withstanding extreme high energy electrical discharges so that large mammalian hearts, such as equine heart, can be defibrillated without thermal injury. In the preferred embodiment, the high energy electrodes measure 10 cm or more and are of size no greater than 24 French (8 mm). The devices can be equipped with a single built-in cable that connects both high energy electrodes on the two catheters by means of a single one piece connector that is customized and fashioned to connect to defibrillators readily found in the field. The devices can also be equipped with a single built in cable that connects both distal tips of the catheters and the low energy electrode on the catheters onto a single one piece connector that is customized and fashioned to connect to defibrillators that are readily found in the field.

The catheter is otherwise designed with normal design attributes known in the art to enhance handling and guidance by use of a braided, torqueable body. Preferably, the indwelling device is capable of being oriented by mechanical deformation at a specific location along its length. For example, the mechanical deformation can occur at or beyond the distal section of the device and the mechanically active section can be further equipped with a flexible ultrasound enhancing sub-system, the sub-system being contained within the catheter and being flexible enough to also deform. The mechanical deformation can occur before the device is inserted into patients such that a pre-set curve on distal end of the catheter is malleable and can be adjusted prior to insertion.

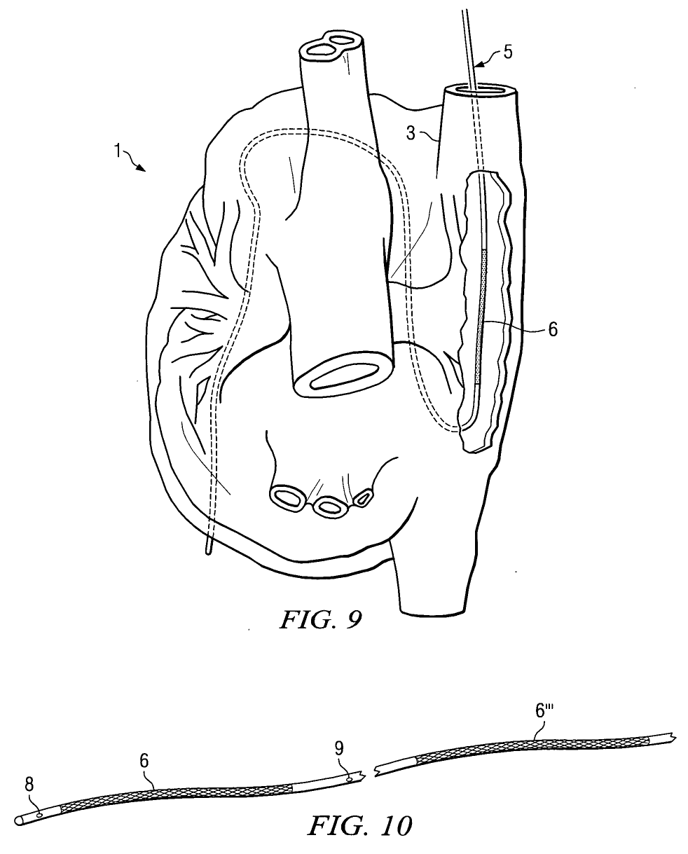

FIGS. 9 and 10 of the drawings show an alternative configuration of the catheter-based system of the invention that uses a single catheter, rather than two. The catheter is shown in greater detail in FIG. 10 and is basically of the same design as the device shown in FIG. 2, with the addition of another high energy electrode 6′. Also, the device shown in FIG. 10 is provided with an additional pressure sensing location 9 just past the first high voltage electrode to ensure this electrode is advanced well past the pulmonary valve and into the left pulmonary branch. The second high voltage electrode can be fashioned of sufficient length so as to be correctly positioned, or made position insensitive, once the distal high voltage electrode is properly positioned in the heart.

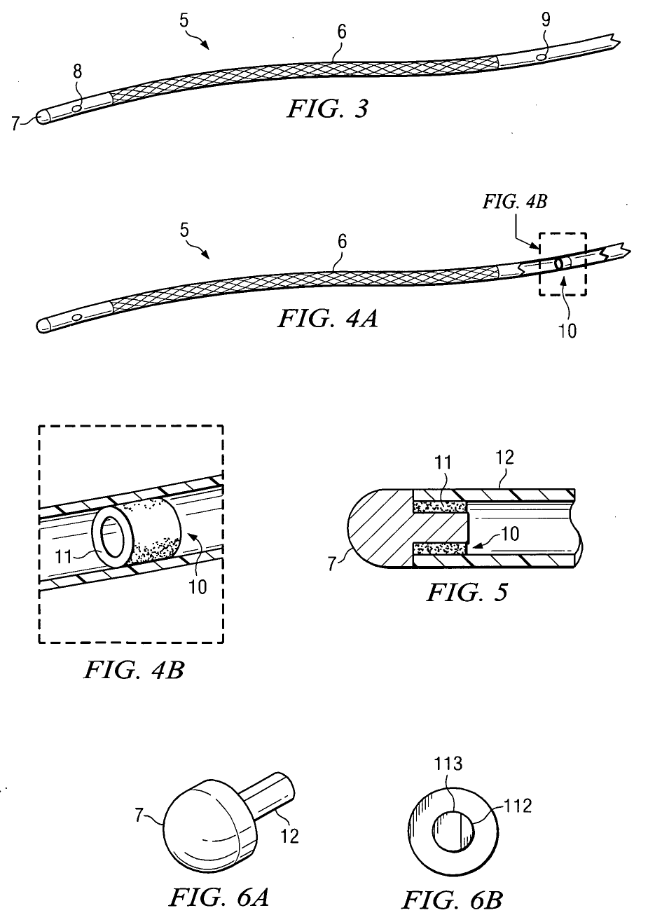

An additional preferred embodiment of the present invention is the use of two catheter-based pressure sensing means. FIG. 3 shows a catheter equipped with two pressure sensing means (8, 9). One sensor (9), is located distal to the high surface electrode and another sensing port (8) is located proximal to the high surface electrode (6). The dual sensor design allows the use of pressure gradients to improve placement of the catheter into the pulmonary artery or other body lumen or organ(s) separated by valve(s). The first sensor (8) disposed in front of the high surface electrode (6) senses the leading edge of the catheter environment. The second sensor (9) behind the high surface electrode senses the environment behind the high surface electrode (6). One preferred embodiment of the present invention is the use of the dual pressure sensing design to help navigate a catheter into the pulmonary artery.

As shown in the associated drawings, the device of the invention can be fashioned with a lumen hole located on the side of catheter, the hole being coupled to an isolated lumen and positioned between 1 mm to 50 mm from the distal end of catheter. The device can also be fashioned with two lumen holes on the side and/or tip of device but located to capture or frame the high surface electrode, one of the lumen holes being at least 1 mm distal of the high surface electrode and the second lumen hole being at least 1 mm proximal to the high surface electrode. The lumen holes can be coupled to independent lumen conduits for hydraulic circuit isolation. A selected lumen hole can be coupled to a common conduit, whereby an average pressure gradient between lumens is observed. The selected lumen hole can be coupled to a common conduit that can be made selectively active to either lumen hole by the use of a telescoping tube that can either open or close either lumen by either blocking or allowing one, none or both of the lumen holes to stay open. Preferably, the conduits connecting the distal end lumen holes are terminated at the proximal end of the device by way of a Luer Lock, or similar fitting.

Depth markers in the form of thin and thick lines can also be applied to the circumference of the catheter. For example, the depth markers can be provided in 25 centimeter increments, where each thin line demarcates a 25 mm displacement, each thick line demarcates a 50 cm displacement, and a line that indicates a location where a curve arc faces inward, so that the user can see and use the mark for additional guidance.

The devices can be ultrasound/echosonograph enhanced, so that visualization is easier, by using sound reflective markers (10), as shown in FIG. 4. The marker (10) is a composite structure of rigid or flexible plastic, epoxy or other adhesive that is used to bind together particles made of glass, ceramic, metal or clay and geometrically ideally suited for sound reflection. The marker (10) is equipped with the composite structure (11) (see FIG. 4B) installed at strategic locations along the catheter. The use of a composite structure marker (10) is also adaptable for use as a combination component for the catheter assembly. FIG. 5 shows that one possible embodiment is the use of adhesives to bind the reflective material, another of its uses is to bond catheter components together. The metallic distal tip (7) is bonded to the elastomeric or plastic catheter body (12) using a composite material composed of items 10 and 11 with item 11 being a bonding adhesive.

The devices can also be made so that directional orientation can be optimized using a composite material and specially machined metal parts. FIGS. 6A and 6B show one possible embodiment where the component being enhanced is the distal tip (7) of the catheter. In this version of the invention the stem is cut so that the metal it is fabricated from includes a “D” shape (113 in FIG. 6B). The stem is then completed to its intended design, a column, by using non-sound reflective material 112, such as, but not limited to, plastic or epoxy. The finished component will reflect an image that has distinct plane differentiation based upon the fact that in one plane the stem is seen as round and in another plane the “D” shape makes the image asymmetrical. The addition can also be made on the distal end or any other portion of the catheter where orientation is important.

The devices of the invention can conveniently be packaged as a set of catheters to be used in a single patient and inclusive of valves, suture straps, introducers and other sterile materials required to treat a patient, so that ease of use is achieved.

The use of the method and preferred device will now be described. A catheter equipped with sound enhancing components as taught above, catheter based pressure sensors and mechanical displacement markers or measuring system, augmented in some cases with ultrasound images, is used to form a quasi-visual-sensory-algorithm (Q-VSA). 14. In one preferred form of the invention, the Q-VSA algorithm is obtained from a device in the form of a system which is constructed into a single transportable unit that can be field ready such that veterinary medicine and military applications can be facilitated, thereby overcoming shortcomings of more cumbersome X-ray based imaging systems.

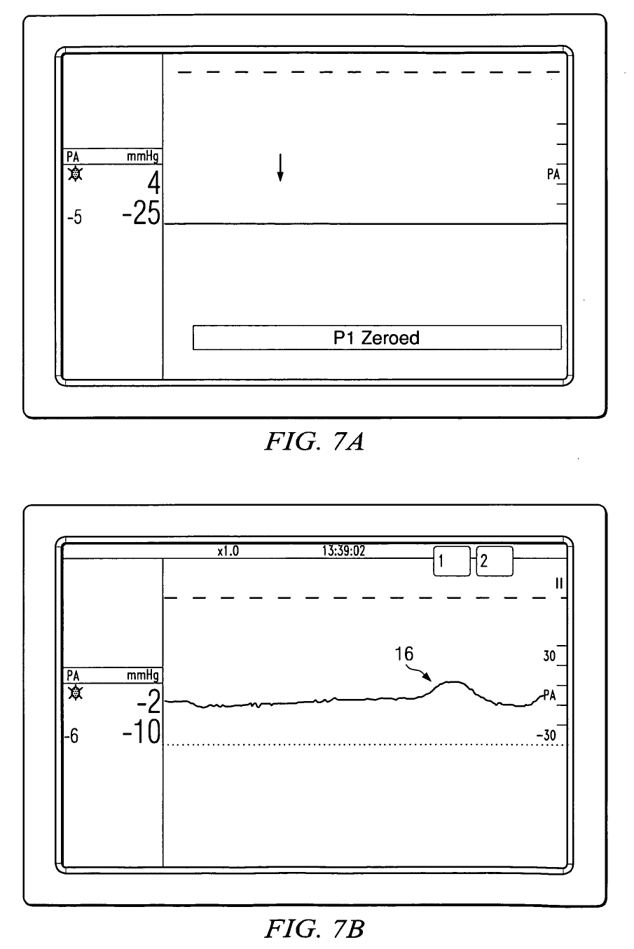

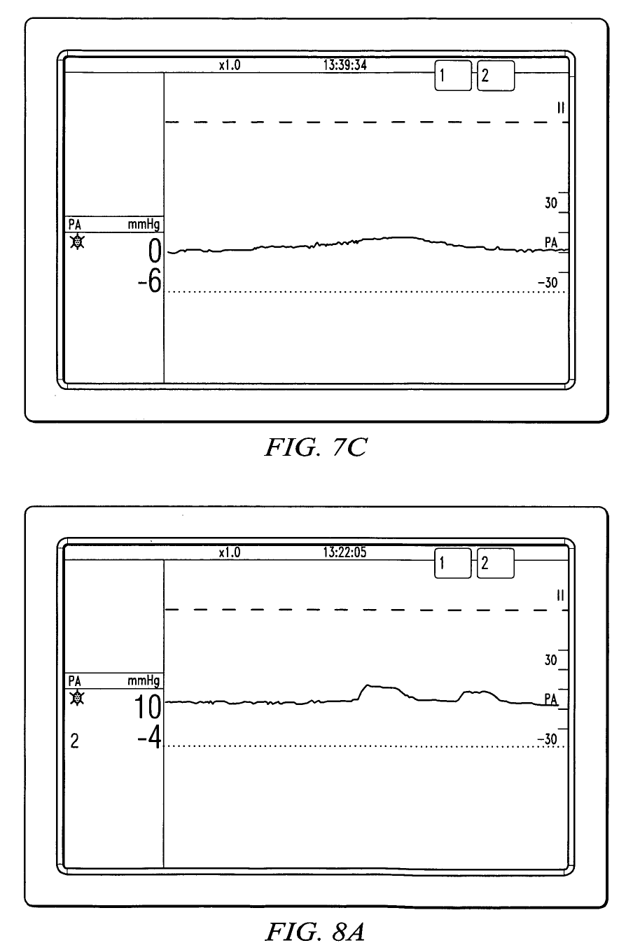

FIGS. 7A-7E shows the images of an actual equine case being performed. The equine atrial fibrillation treatment process is done in three steps consisting of placing one catheter into the left pulmonary artery (LPA) then placing a second catheter into the right atrium (RA) and finally delivering electrical energy. The process is started by insertion of the first catheter, the PA catheter, into the jugular vein of an equine and then advancing the catheter about 20 centimeters with the curved section of catheter pre-disposed so that it faces downward. The catheter mounted pressure sensor is then zeroed (FIG. 7A) to the environment since absolute pressure measurements are not required but instead pressure change (gradients) are used.

The catheter is then advanced with care taken so the catheter does not twist during insertion so the curved section remains pointed downwards. The catheter will move into the right atrium and then the curve will cause the catheter's distal end to advance of the catheter into the right ventricle. The use of mechanical displacement markers and/or measurement will be used to monitor advancement. The catheter mounted pressure sensor at the distal end of catheter will provide internal (indwelling) sensory information (FIG. 7B) showing when the catheter is within the right ventricle. The pressure gradient, shown in FIG. 7B, indicates the catheter distal end has entered the ventricle.

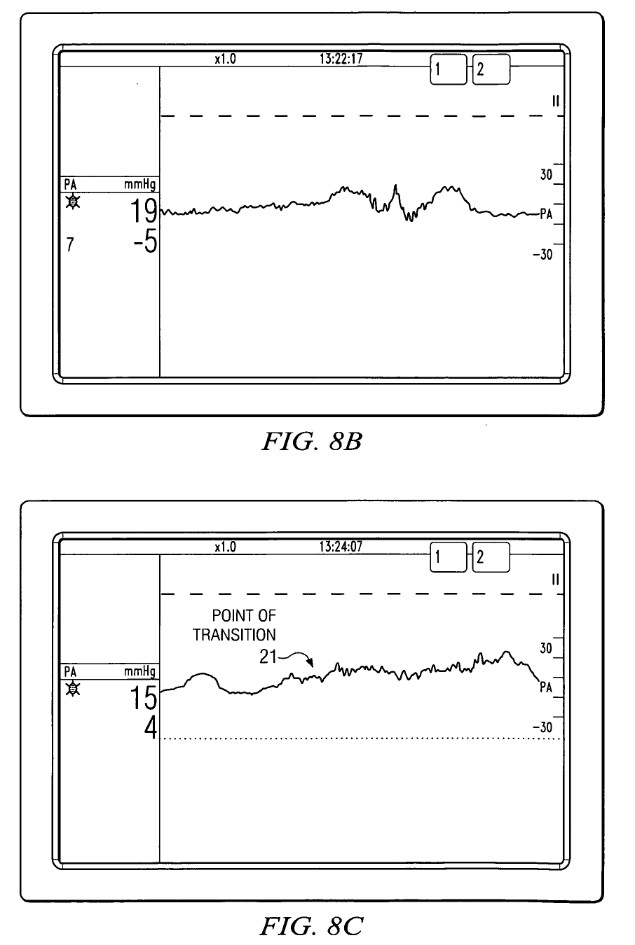

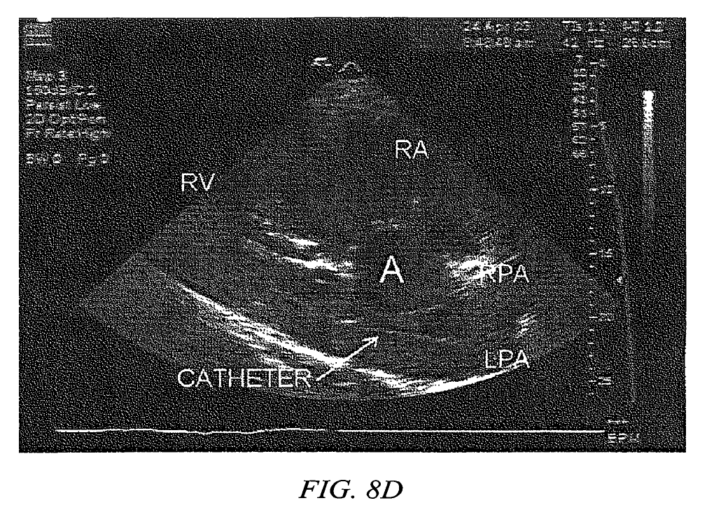

The catheter is advanced into the right ventricle as shown in FIGS. 7A-7C and both mechanical displacement and pressure gradients (see FIG. 8A) are used to confirm status. The catheter is then further advanced into the right heart outflow tract (FIG. 8B) and finally into the PA with confirmation of placement made using pressure gradient change (FIG. 8C). The transition of catheter from Right Ventricle to PA is obvious when observed using the pressure gradient 21. The catheter is then further advanced into the left pulmonary branch using ultrasound (see FIG. 8D) as the primary guidance system. The catheter is manipulated by use of the torqueable body or deflectable distal end into the left pulmonary branch so that both the left and right atrial muscle mass are captured with the shock vector. The RA catheter is then inserted in similar fashion to the PA catheter, through the jugular vein and just into the right ventricle. The placement of the RA catheter is completed by simply pulling back the RA catheter until the ventricular pressure gradient (16 in FIG. 7B) disappears (see FIG. 7C), which indicates the catheter's distal end and high surface electrode is within the right atrium.

The method can preferentially allow, at the option of clinician, the high surface electrode in RA to rest along the upper and latter walls of the right atrium since the stored energy of the catheter distal end will create outward mechanical force, pushing the catheter against the heart muscle. The RA catheter therefore rests against the lateral free wall of right atrium and also against the atrial septum.

The process herein disclosed is further enhanced by the use of the dual pressure system shown in FIG. 3, because the second pressure sensor (9) mounted on the catheter provides confirmation of the location of the proximal end of the high surface electrode (6) in the PA to insure that catheter is ideally positioned prior to cardioversion. The second sensor would ideally be used to ensure that the high surface electrode is fully inside the pulmonary artery and well above the pulmonic valve. This will ensure that no ventricular muscle mass is affected by the depolarizing current and help to maximize the volume of atrial muscle mass depolarized.

Additionally, in areas where pressure gradients are not useful or cannot be used to guide device, an electrical signal gradient can be used whereby a reference signal (ECG or the like) from a reference set of electrodes (located on a single or multiple devices positioned indwelling, external or combination thereof) is used to gather a baseline reference signal. The reference signal is then used to guide a separate catheter/lead into position as the electrical signal gradients are used for navigation.

In one situation, two devices, each having one or more pairs of electrodes, is positioned near a known signal source within the body, such as the bundle of an HIS, and the second catheter is positioned into the left pulmonary branch by using a comparison of electrical signal. The electrical signal taken from a catheter/lead mounted sensing pair(s) is different enough when measured in the right and left pulmonary branches, such that the signals can be used to confirm anatomical position and or depth within body organ or lumen. The reference electrical sensing pairs as well as the electrical guidance sensing pairs can all be mounted on a single catheter/lead in some cases.

An invention has been provided with several advantages. The present invention teaches the use of several internal and external based measurements and ultrasound images that can be used to navigate catheters deep into the heart. The measurements are used together to create a quasi-visual-sensory-algorithm (Q-VSA). The system relies on several inputs provided to a clinician that originate from both external and internal sources. The external source is an ultrasound system image of the anatomy displayed as a cross sectional view. The internal input comprises intra-luminal pressure gradients taken at or near the distal tip of catheter, with an optional second catheter based input being electrical signals taken at or near the tip.

While the invention has been shown in only one of its forms, it is not thus limited but is susceptible to various changes and modifications without departing from the spirit thereof.

What is claimed is:

1. A method of guiding a catheter within a mammalian body, the method comprising the steps of:
positioning a catheter into a deep body lumen or organ using an external ultrasound image as a principal guidance system but also in conjunction with pressure gradients taken from an associated internally located device having sensors or pressure lumens mounted thereon;
using the ultrasound image and pressure gradients to guide the catheter into a desired position within the body.

2. The method of claim 1, wherein the ultrasound image which is used to guide the catheter into a specific location within the body uses anatomical distinctions and guidance decisions made from specific physiological measurements made by way of the catheter based sensors.

3. The method of claim 2, further comprising the use of multi-plane ultrasound scans done using external means and pressure gradient measurements and depth markers and depth sensors mounted on the device to form a quasi-visual-sensory-algorithm (Q-VSA);

4. The method of claim 3, wherein the algorithm of depth, image and pressure gradients is used to create an expected series of measurements that provide sufficient affirmation for placement of devices within targeted anatomical locations within the body.

5. The method of claim 4, wherein a set of indwelling devices, working in tandem, are used to generate an electric field between electrodes mounted on the two devices, the devices being guided and preferentially positioned within the heart using the Q-VSA method.

6. The method of claim 5, wherein the mammalian heart is human having two sets of chambers consisting of a left and right atrium and left and right ventricles.

7. The method of claim 6, wherein the mammalian heart is an equine animal.

8. The method of claim 6, wherein the mammalian heart is a canine animal.

9. The method of claim 6, wherein the mammalian heart is a feline animal.

10. The method of claim 6, wherein the mammalian heart is a bovine animal.

11. The method of claim 6, wherein the mammalian heart is a porcine animal.

12. The method of claim 6, wherein the mammalian heart is a mastodon animal.

13. The method of claim 6, wherein the mammalian heart is that of mammal weighing greater than about 1 kilogram.

14. The device of claim 6, wherein the Q-VSA algorithm is obtained from a device in the form of a system which is constructed into a single transportable unit that can be field ready such that veterinary medicine and military applications can be facilitated, thereby overcoming shortcomings of more cumbersome X-ray based imaging systems.

15. The device of claim 14, wherein the indwelling device is equipped with a semi-flexible high surface electrode capable of withstanding extreme high energy electrical discharges so that large mammalian hearts, such as equine heart, can be defibrillated without thermal injury.

16. The device of claim 15, wherein the indwelling device is capable of being oriented by mechanical deformation at a specific location along its length.

17. The device of claim 16, wherein the indwelling device is further equipped with ultrasound/echosonograph image enhancing attributes selected from the group consisting of bonding adhesives and additional cast markers that contain hollow or solid micro spheres made of glass, ceramic, plastic, metal or clay.

18. The method of claim 17, wherein the mechanical deformation occurs at or beyond the distal section of the device and the mechanically active section is further equipped with a flexible ultrasound enhancing sub-system, the sub-system being contained within the catheter and being flexible enough to also deform.

19. The method of claim 18, wherein the mechanical deformation occurs before the device is inserted into patients such that a pre-set curve on distal end of the catheter is malleable and can be adjusted prior to insertion.

20. The method of claim 18, wherein the device is fashioned with a lumen hole located on the side of catheter, the hole being coupled to an isolated lumen and positioned between 1 mm to 50 mm from distal end of catheter.

21. The method of claim 18, wherein the device is fashioned with two lumen holes on the side and/or tip of device but located to capture or frame the high surface electrode, one of the lumen holes being at least 1 mm distal of the high surface electrode and the second lumen hole being at least 1 mm proximal to the high surface electrode.

22. The method of claim 21, wherein the lumen holes are coupled to independent lumen conduits for hydraulic circuit isolation.

23. The method of claim 21, wherein a selected lumen hole is coupled to a common conduit and an average pressure gradient between lumens is observed.

24. The method of claim 21, wherein the selected lumen hole is coupled to a common conduit that can be made selectively active to either lumen hole by the use of a telescoping tube that can either open or close either lumen by either blocking or allowing to stay open one, none or both of the lumen holes.

25. The method of claim 24, whereby the conduits connecting distal end lumen holes are terminated at the proximal end of the device by way of a Luer Lock, or similar fitting.

26. The method of claim 25, wherein depth markers in the form of thin and thick lines are applied to the circumference of the catheter in 25 centimeter increments and each thin line demarcates a 25 mm displacement, each thick line demarcates a 50 cm displacement, and a line that indicates a location where a curve arc faces inward is also applied, so that the user can see and use the mark for additional guidance.

27. The method of claim 26, wherein the devices are packaged as a set of catheters to be used in a single patient and inclusive of valves, suture straps, introducers and other sterile materials required to treat a patient, so that ease of use is achieved.

28. The method of claim 27, wherein the high energy electrodes measure 10 cm or more and are of size no greater than 24 French (8 mm).

29. The method of claim 28, wherein the devices are equipped with a single built-in cable that connects both high energy electrodes on the two catheters by means of a single one piece connector that is customized and fashioned to connect to defibrillators readily found in the field.

30. The method of claim 28, wherein the devices are equipped with a single built in cable that connects both distal tips of the catheters and the low energy electrode on the catheters onto a single one piece connector that is customized and fashioned to connect to defibrillators that are readily found in the field.

31. A method of guiding a catheter within a mammalian body, the method comprising the steps of:
positioning a catheter into a deep body lumen or organ using an external ultrasound image as a principal guidance system but also in conjunction with pressure and electrical gradients taken from an associated internally located device having electrical sensors or pressure sensors mounted thereon;
using the ultrasound image and pressure gradients the catheter can be guided to areas where pressure gradient disparity and anatomical location can be correlated position within the body.
